(19) United States
(12) Patent Application Publication
(10) Pub. No.: US 2025/0177894 A1
(43) Pub. Date: Jun. 5, 2025

(54) FILTER DEVICE

(71) Applicant: Bird Healthcare Pty Ltd, Bayswater (AU)

(72) Inventors: Nicholas James Bird, Melbourne (AU); Timothy Boult, Bayswater (AU); Neale Gover, Bayswater (AU)

(21) Appl. No.: 19/048,698

(22) Filed: Feb. 7, 2025

Related U.S. Application Data
(63) Continuation of application No. PCT/AU2023/050740, filed on Aug. 8, 2023.

(30) Foreign Application Priority Data
Aug. 8, 2022 (AU) 2022902220

(51) Int. Cl. B01D 35/30

(52) U.S. Cl. CPC B01D 35/306; B01D 35/301; B01D 2201/305

(57) ABSTRACT

The invention provides a filter device 10 for a breathing device, the filter device 10 comprising: a housing 12 with a wall 13 made from a paper material and defining a volume, two openings 14, 16 in the wall 13 that communicate with the volume and a filter mounting portion 18 configured for mounting a filter media in the volume between the openings 14, 16. The invention also comprises an adaptor to connect a filter device 10 to a breathing device, the adaptor also made from a paper material.

### CROSS-REFERENCES TO RELATED APPLICATIONS

This application is a continuation application of International Patent Application No. PCT/AU2023/050740 entitled “A FILTER DEVICE,” filed on Aug. 8, 2023, which claims priority to Australian Patent Application No. 2022902220, filed on Aug. 8, 2022, each of which are herein incorporated by reference in their entirety for all purposes.

### FIELD

The invention relates a filter device for a breathing device.

The invention also relates a filter assembly comprising a filter device.

The invention also relates to a method of manufacturing a filter device.

The invention also relates to an adaptor for use with a filter device.

The invention relates to a combination of a filter device and an adaptor coupled together.

The invention also relates to a breathing device that comprises a filter assembly.

### BACKGROUND

A breathing device is a device that a user breathes through to medically assist the user. Breathing devices include diagnostic devices that measure aspects of a user's breathing cycle, such as spirometers, and devices that help the user breathe, for example resuscitation bags, ventilators, face masks, biPAP machines and CPAP machines.

A filter device is often positioned between a user and a breathing device to filter particulates from an airflow passing through the breathing device to the user. The filter device typically includes a housing having a passage through which the airflow passes, and a filter media located in the channel configured to remove particulates including bacteria and viruses from the airflow.

The filter devices, although often being made from recyclable material, typically polymer, are difficult to recycle and are often disposed of in landfill or incinerated. Neither of these disposal methods is ideal. The filter devices can take decades or even hundreds of years to decompose in landfill and the exhaust gasses produced from incineration are harmful to people and to the environment.

It is desirable to ameliorate at least one of the above disadvantages or to at least provide a useful alternative.

The above description is not an admission of the common general knowledge in Australia or elsewhere.

### SUMMARY

The invention provides separately or in combination a filter device for a breathing device and an adaptor for use with a filter device.

The invention provides a filter device for a breathing device, the filter device comprising: a housing with a wall made from a paper material and defining a volume, two openings in the wall that communicate with the volume, and a filter mounting portion configured for mounting a filter media in the volume between the openings.

The filter device is configured such that in use, with the filter media mounted in the filter mounting portion in the housing, a user can inhale through one of the openings, such that air is drawn through the other of the openings to flow past the filter media and into the user's lungs. When air is drawn through the filter media, particulates in the airflow, including bacteria and viruses, can be filtered therefrom.

In the context of this application, the term “paper material” includes any material that comprises plant fibers, for example cellulose. The plant fibers may be agglomerated or woven to provide structure. Sheets of plant fibers may be arranged in layers to provide structure. The paper material may comprise plant fibers and water, for example as a pulp, which is then dried to provide a solid material. The plant fibers may be derived from trees or from recycled paper.

In one example, the housing is formed from pulp in a mold under negative pressure, i.e., a vacuum.

A person skilled in the art would appreciate that the housing should have sufficient strength to maintain structural integrity whilst the user is generating a pressure in the housing as a result of the user inhaling or exhaling through one of the openings.

The filter mounting portion is configured to hold the filter media relative to the housing.

The filter mounting portion may be configured to restrict at least one of lateral, rotational, translatory or bending (i.e., deflection) movement of the filter media relative to the housing. The filter mounting portion may produce a retaining force that grips the filter media.

A person skilled in the art would appreciate that the retaining force should be sufficient to hold the filter media in place whilst the user is generating a pressure in the housing as a result of the user inhaling or exhaling through one of the openings.

An advantage of the present invention is that, because the housing is made from a paper material, it is more readily disposable. Paper material quickly decomposes in compost or in landfill when compared with plastic alternatives. Paper material is also more combustible and produces fewer noxious exhaust gasses as a result of incineration when compared with plastic alternatives.

An advantage of the filter mounting portion is that it makes it possible for the filter media to be secured against movement relative to the housing. This makes it possible to enhance the performance of the device by maintaining adequate sealing between the housing and the filter media.

The filter mounting portion may comprise a projection that extends away from the wall inwardly into the volume and defines a support for the filter media.

The filter mounting portion may comprise at least one pair of opposed projections which each extend inwardly into the volume from opposed parts of the wall and are configured to sandwich the filter media therebetween and thereby hold the filter media in position.

An advantage of the projection is that it not only serves as filter mounting portion but also acts as a stiffening rib. This is particularly important for paper materials which are typically less rigid than polymers.

The projection may be integrally formed in the wall of the housing.

An advantage to integrally forming the projection with the wall is that additional fasteners are not required and therefore manufacturing costs are reduced.

However, the projection may be a separate component which, as part of a process of assembling the filter device, is attached to the wall of the housing to form an assembly.

The projection may be a ridge in the wall. The ridge may extend, completely or partially, around at least one of the openings. The groove or ridge may extend, completely or partially, around both openings.

The wall may be indented inwardly into the volume to form the projection.

The filter mounting portion may comprise a plurality of projections.

The plurality of projections may be arranged around at least one of the openings. The plurality of projections may be arranged around both openings.

An advantage of the above arrangement of projections is that it serves to align/locate the filter media in the correct position. This enhances the performance of the device by ensuring correct sealing between the housing and the filter media.

The housing may comprise a first shell half defining a part of the wall and a second shell half defining another part of wall that are releasably coupled together.

An advantage of releasably coupling the first and second shell halves is that one of the first and second shell halves can be replaced without replacing the other. This is particularly important for housings made from paper material, as one of the first and second shell halves may deteriorate prior to the other.

A further advantage is that the filter media can be accessed and replaced.

The filter device may comprise corresponding engagement elements for releasably coupling together the shell halves.

The first shell half may be dished shaped.

The second shell half may be dished shaped.

In the context of this application, the term “dished shaped” refers to a shape that has the appearance of a dish. In other words, a circular shape when viewed in a first plane that is also concave/convex when viewed in a second plane that is perpendicular to the first plane.

However, the first and/or second shell halves may be any other shape. For example, box shaped, cone shaped, or frustoconical shaped.

The engagement elements may secure one shell half relative to the other shell half.

The engagement elements may be integrally formed in the wall of the housing.

Alternatively, the engagement elements may be formed from separate components that are then attached to walls of the housing as part of a process of assembling the filter device.

The engagement elements may comprise a male element and a female element.

In one example, one of the shell halves has a male element and the other of the shell halves has a corresponding female element. The male element may be insertable inside the female element in a friction fit arrangement.

The male element can be configured to be frictionally inserted into the corresponding female element.

The female element can be configured to frictionally receive the corresponding male element.

An advantage of the friction fit arrangement is that a small number or no other fasteners are required for assembly. In addition, fewer or no machines are required for assembly. For example, the fitting can be done by hand and by the user. In contrast, prior art devices require machines to assemble because they involve processes such as crimping, adhesives or ultrasonic welding.

The paper material may have a rough surface finish that promotes frictional resistance when like surfaces meet as compared with rough on smooth surfaces or smooth on smooth surfaces. It is advantageous to provide frictional resistance at the friction fit arrangement of the male and female elements in order to maintain structural integrity of the housing when under pressure.

In one example, the male element is a lip. The lip may be defined by a contour of, or curvature in, the wall. The lip may extend, completely or partially, around the perimeter of one shell half.

In one example, the female element is a channel. The channel may be defined by a contour of, or curvature in, the wall. The channel may extend, completely or partially, around the perimeter of the other shell half.

It is also envisaged that the engagement elements may be any element capable of securing one shell half relative to the other shell half. Examples of engagement elements include permanent fasteners (such as rivets), reusable fasteners (such as hook and loop fasteners, i.e., Velcro®), and adhesives.

The filter mounting portion may comprise a first projection on the first shell half and a second projection on the second shell half, each projection extending inwardly into the volume from the respective parts of the wall, the projections being configured to frictionally engage a filter media therebetween when the first and second shell halves are coupled together. Suitably, the filter media is sandwiched between the projections when the first and second shell halves are coupled together.

The filter media may be clamped between the projections when the first and second shell halves are coupled together.

The first projection may comprise a plurality of projections.

The second projection may comprise a plurality of projections.

The plurality of projections may be arranged such that, the filter media is supported around its perimeter.

Suitably, the filter media is clamped around its perimeter when the first and second shell halves are coupled together.

The first shell half may comprise an inlet configured for attachment to a breathing device. The inlet is one of the above-described openings of the filter device. In one example, the inlet is a spigot arrangement that projects from the first shell half. The inlet may have a circular cross-sectional shape. However, it is also envisaged that the inlet may have any suitable cross-sectional shape, for example: rectangular, triangular, trapezoidal, or oval.

The second shell half may comprise an outlet configured to conform to a mouth of a user. The outlet is one of the above-described openings of the filter device. In one example, the outlet is a spigot arrangement that projects from the second shell half. The outlet may have an oval cross-sectional shape. However, it is also envisaged that the outlet may have any suitable cross-sectional shape, example: rectangular, triangular, trapezoidal, or oval.

The first shell half may be shaped such that a plurality of first shell halves can be stacked in a nested arrangement.

The second shell half may be shaped such that a plurality of second shell halves can be stacked in a nested arrangement.

An advantage of stacking the first and second shell halves in a nested arrangement is that it can reduce packing volume, which may contribute towards a reduction in shipping costs.

The invention also provides a filter assembly comprising a filter device as previously described and a filter media mounted to the filter mounting portion within the housing.

The filter media may be a barrier that provides restricted passage of particulates, including bacteria and viruses, therethrough but allows relatively unrestricted passage of air therethrough.

The filter media may provide between 90% and 100% bacterial/viral filtration. Optionally, the filter media may provide around 99.9999% bacteria/viral filtration.

The filter media may provide a resistance of between 0.5 and 1.0 cm H2O/L/s at a flow of 12 L/s. Optionally, the filter media may provide a resistance of approximately 0.71 cm H2O/L/s at a flow of 12 L/s.

The filter media may be any suitable shape.

For example, the filter media may be disc shaped.

The filter media may be porous.

The filter media may be electrostatic.

The filter media may be hydrophobic.

The invention also provides a method of manufacturing a filter device for a breathing device, the filter device comprising: a housing having a wall made from a paper material and defining a volume, two openings in the wall that communicate with the volume and a filter mounting portion configured for mounting a filter media in the volume between the openings, the method of manufacturing comprising: molding a paper material into a shape of the housing in a mold; drying the housing; and removing the housing from the mold.

The invention also provides an adaptor for use with a filter device, the adaptor comprising an adaptor housing having a first end that is configured for attachment with the filter device, a second end configured for attachment with a breathing device, and an internal passage that extends between the first and second ends, such that, in use, airflow is permitted between the breathing device and the filter device through the internal passage.

An advantage of the adaptor is that it allows the filter device to be used with a greater variety of different breathing devices. For example, some breathing devices have a bayonet style connection comprising a projection that is intended to cooperate with a track on a component that is to be connected to the breathing device. The adaptor can be said to increases the flexibility of the filter device.

The adaptor housing may be made from a paper material.

The paper material may be the same paper material that forms the filter device to provide the same advantages as previously described.

The adaptor housing may be made from any suitable material. For example, polymer or stainless steel.

The second end may have an engagement portion that allows releasable connection with the breathing device.

Releasable connection between the second end and the breathing device may involve an insertion and twisting motion.

The housing may comprise a side wall defining an internal surface and an external surface.

The side wall may be cylindrical.

The engagement portion may comprise a track or a projection on an internal or an external surface that cooperates with a corresponding track or projection on the breathing device.

The track may be an opening that extends all through the side wall.

The track may be a recess into an internal surface the side wall.

The track may be a projection that cooperates with a corresponding groove on the breathing device.

In use, the first end of the adaptor may be connected to the inlet spigot of the filter device. The first end may be connected to the inlet spigot via a friction fit. For example, the first end may be configured (shaped and/or dimensioned) such that it can be inserted into the opening of the inlet spigot in a force fitting relationship. In another example, the first end may be configured (shaped and/or dimensioned) such that the spigot can be inserted into the internal passage of the adaptor in a force fitting relationship.

The track may be curved or bent such that releasable connection between the second end and the breathing device involves an insertion and twisting motion.

An advantage of this two-step connection process is that it provides additional security over other methods that involve only one of an insertion or a twisting motion.

The invention also provides the filter device as described coupled to the adaptor as described.

The invention also provides the filter assembly as described coupled to the adaptor as described.

The invention also provides a combination of the above-described filter device and the above-described breathing device operatively coupled together.

The invention also provides a breathing device that comprises the above-described filter assembly.

The invention also provides a breathing device that comprises the above-described filter assembly and the above-described adaptor.

### BRIEF DESCRIPTION OF THE DRAWINGS

Embodiments of the invention are described further by way of example with reference to the accompanying drawings of which:

FIG. 1 is a top perspective view of the filter device according to an embodiment of the present invention;

FIG. 2 is a bottom perspective view of the filter device of FIG. 1;

FIG. 3 is an exploded perspective view of the filter device according to FIG. 1;

FIG. 4 is a bottom view of in the exploded filter device shown in FIG. 3;

FIG. 5 is a top view of the filter device of FIG. 1;

FIG. 6 is a bottom view of the filter device of FIG. 1;

FIG. 7 is a side view of the filter device of FIG. 1;

FIG. 8 is a view of filter device of shown in FIG. 7, rotated axially by 90 degrees;

FIG. 9 is a top perspective view of a filter assembly, in a disassembled form, according to an embodiment of the invention, the filter assembly comprising a first shell half, a second shell half and a filter media;

FIG. 10 is a sectional view of the first shell half shown in FIG. 9;

FIG. 11 is a sectional view of the second shell half shown in FIG. 9;

FIG. 12 is a flow-diagram showing the method steps involved in manufacturing a filter device according to an embodiment of the present invention;

FIG. 13 is a schematic side view of an adaptor according to another embodiment of the present invention;

FIG. 14 is a schematic top view of the adaptor of FIG. 13;

FIG. 15 is a schematic cross-sectional view of the adaptor of FIG. 13 along the line A-A showing an engagement portion comprising an angled track having a first portion and a second portion arranged orthogonally with respect to each other;

FIG. 16A is the adaptor shown in FIG. 13 with a projection inserted into a first portion of the track;

FIG. 16B is the adaptor shown in FIG. 16A with the projection inserted towards an end of the first portion of the track; and

FIG. 16C is the adaptor shown in FIG. 16A having been rotated such that the projection has travelled towards an end of a second portion of the track.

### DETAILED DESCRIPTION OF SPECIFIC EMBODIMENTS

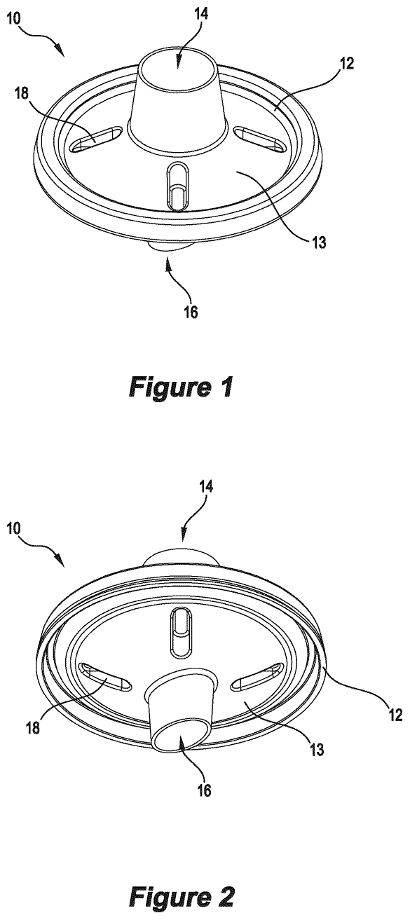

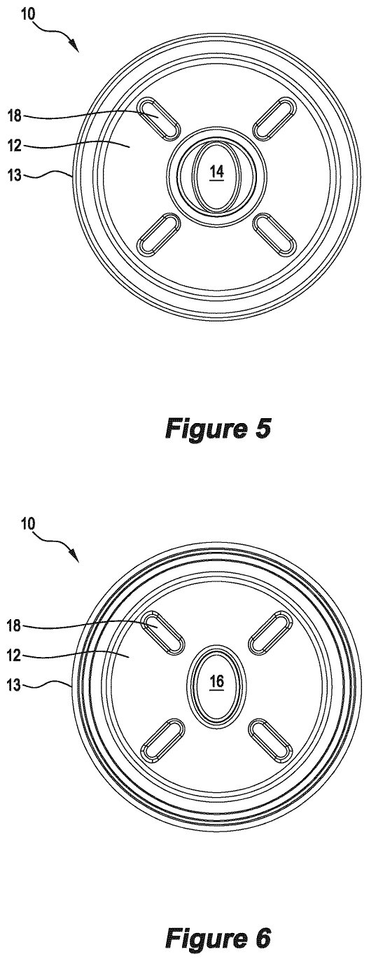

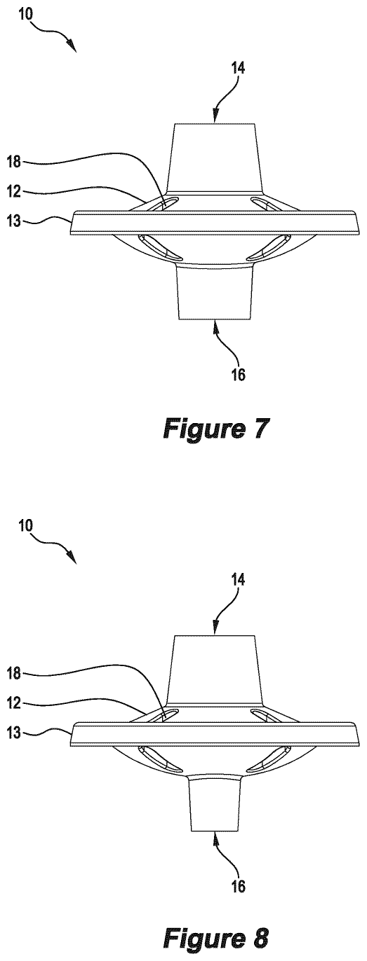

FIGS. 1-8 show a filter device 10 according to an embodiment of present invention.

The filter device 10 comprises: a housing 12 having a wall 13 made from a paper material and defining a volume, the housing 12 having a pair of openings 14, 16 that communicate with the volume and a filter mounting portion 18 configured for mounting a filter media in the volume between the openings 14, 16.

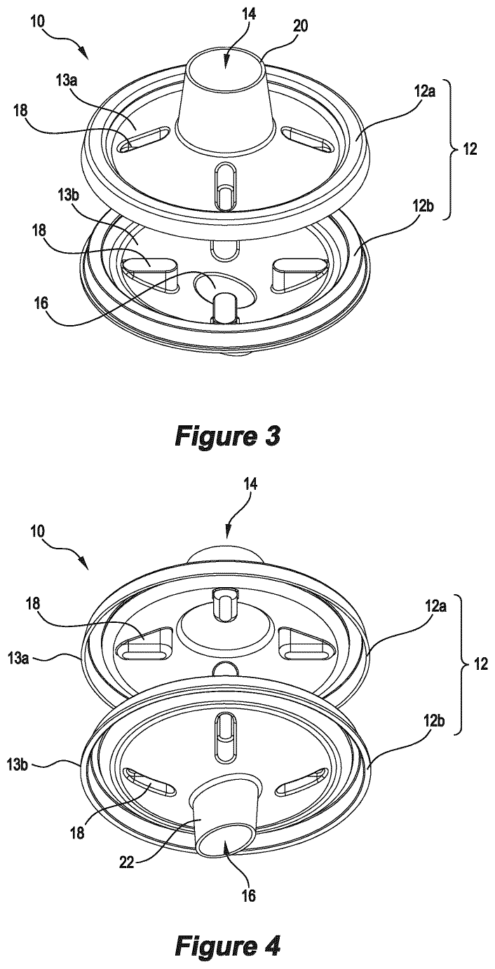

As best shown in FIGS. 3 and 4, the housing 12 comprises a first shell half 12a, that defines a part of the wall 13a, and a second shell half 12b, that defines another part of the wall 13b, that are releasably coupled to each other via corresponding engagement elements. When the first shell half 12a and the second shell half 12b are coupled to each other, they define the housing 12 and the volume bounded by the wall 13.

The filter mounting portion 18 comprises a plurality of projections that extend inwardly into the volume. The projections are defined by the contour of the respective walls 13a, 13b. When the first shell half 12a and the second shell half 12b are coupled together the filter media 25 can be sandwiched between opposing projections 18.

The first shell half 12a comprises an inlet spigot 20 (see FIG. 3) configured for attachment to a breathing device. The inlet spigot 20 comprises an inlet opening 14 which is circular shaped.

The second shell half 12b comprises an outlet spigot 22 (see FIG. 4) configured to conform to a mouth of a user. The outlet spigot 22 comprises an outlet opening 16 which is oval shaped.

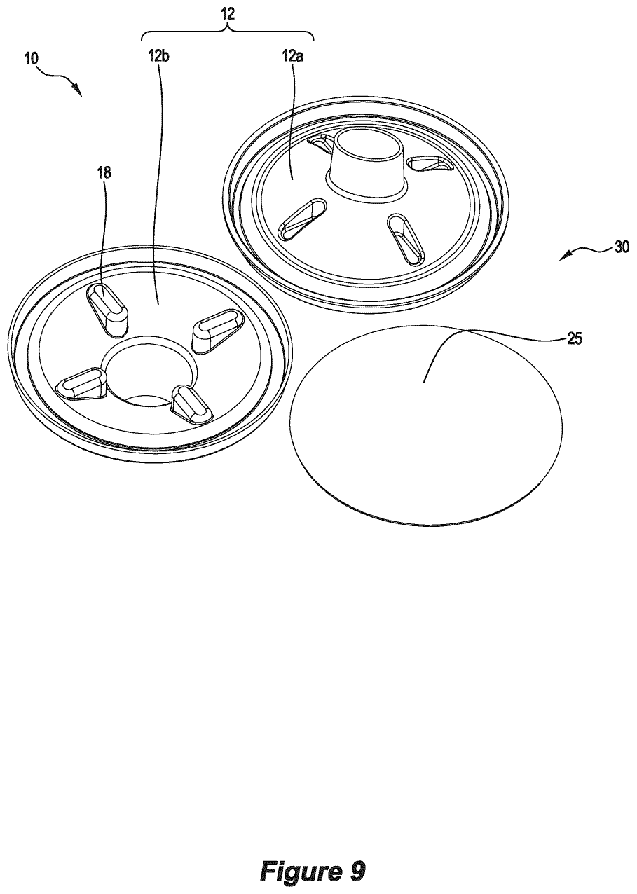

FIGS. 9-11 show a filter assembly 30 which comprises a filter device 10 and a filter media 25. The filter device 10 has the features as previously described and comprises first and second shell halves 12a, 12b.

To assemble the filter assembly 30, the filter media 25 is first positioned on the second shell half 12b so that it is supported by the projections 18. The first shell half 12a is then coupled to second shell half 12b such that the filter media 25 is sandwiched and secured between opposed projections 18 on the first and second shell halves 12a, 12b.

Alternatively, the filter media 25 is first positioned on the first shell half 12a so that it is supported by the projections 18. The second shell half 12b is then coupled to the first shell half 12a to sandwich and secure the filter media 25 between opposed projections 18 in a similar manner as previously described.

It can be appreciated that the filter media 25 can be removed from the filter device 10 by uncoupling the first shell half 12a from the second shell half 12b.

Enabling removal of the filter media 25 from the filter device facilitates ease of replacement of the filter media 25.

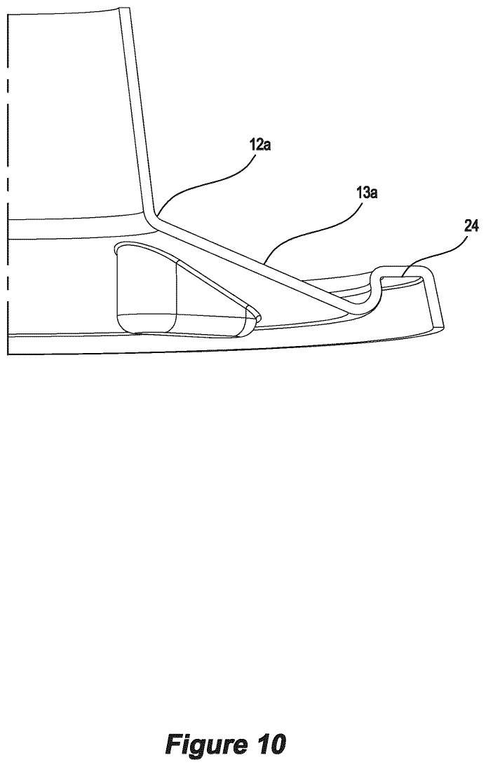

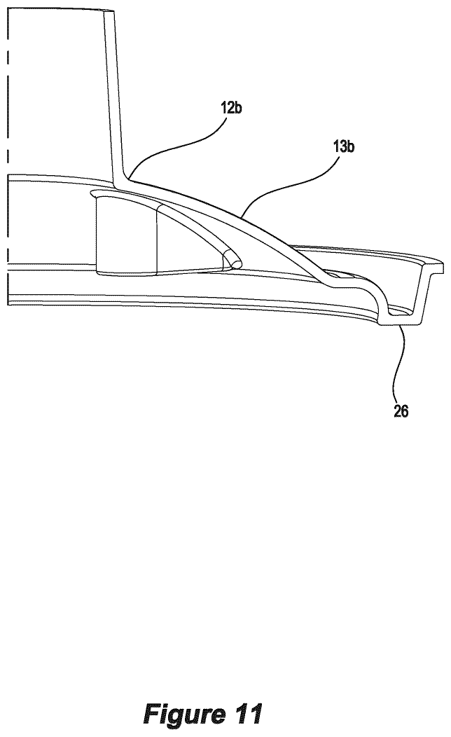

As best illustrated in FIGS. 10 and 11, the first and second shell halves 12a, 12b each comprise an engagement element.

The first shell half 12a comprises a female engagement element in the form of a channel 24. The channel 24 is defined by a contour of the wall 13a, as shown in FIG. 10.

The second shell half 12b comprises a male engagement element in the form of a lip projection 26. The lip projection 26 is defined by a contour of the wall 13b, as shown in FIG. 11.

Both the channel 24 and the lip projection 26 extend completely around the perimeter of the respective shell half 12a, 12b.

The channel 24 is configured (dimensioned and shaped) such that the lip projection 26 can be received within the channel 24 to form a friction fit therebetween to couple the first shell half 12a to the second shell half 12b.

The first shell half 12a is shaped such that a plurality of first shell halves 12a can be stacked in a nested arrangement.

The second shell half 12b is shaped such that a plurality of second shell halves 12b can be stacked in a nested arrangement.

An advantage of stacking the first and second shell halves 12a, 12b in a nested arrangement is that it can reduce packing volume, which may contribute towards a reduction in shipping costs.

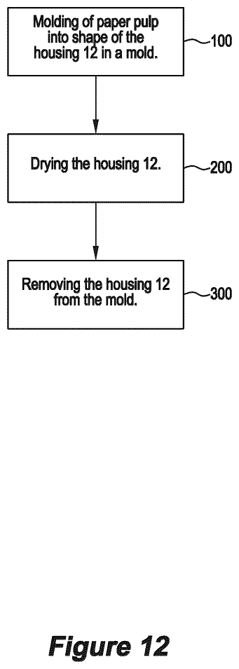

FIG. 12 shows a method of manufacturing a filter device 10 for a breathing device. The method of manufacturing comprises the following steps:
Step 100: molding a paper pulp into a shape of the housing 12 in a mold;
Step 200: drying the housing 12; and
Step 300: removing the housing 12 from the mold.

Each of the shell halves 12a, 12b is produced within a separate mold. The molding is performed under negative pressure, i.e., a vacuum. The mold comprises a porous surface to ensure even distribution of pressure through the mold.

The step of drying the housing is performed in an oven.

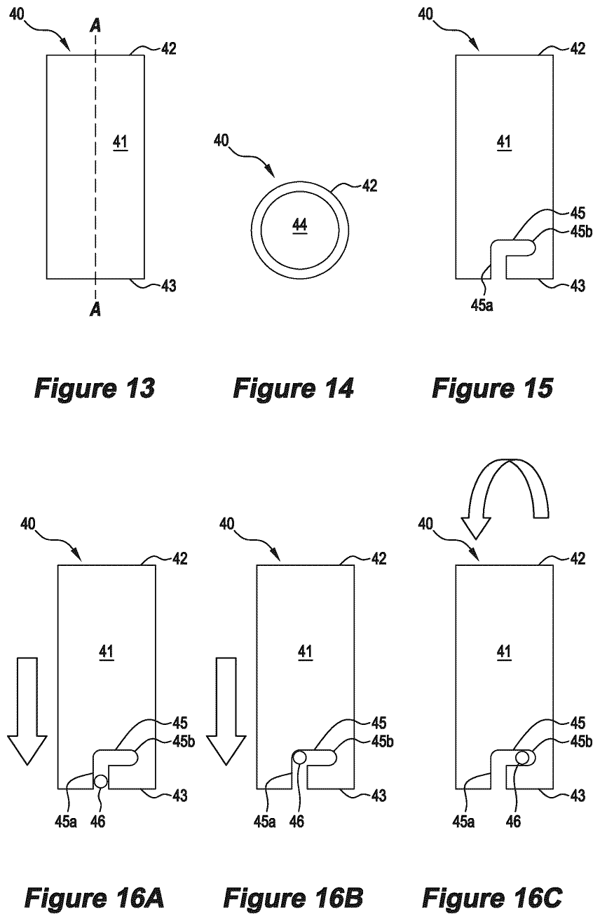

FIGS. 13-16C show an adaptor 40 for use with the filter device 10, as previously described, in accordance with another embodiment of the invention.

The adaptor 40 comprises a tubular housing formed from a cylindrical side wall 41. The housing has a first end 42 that is configured for attachment with the filter device 10, and a second end 43 configured for attachment with a breathing device (not shown). The breathing device may be any suitable breathing device.

As shown in FIG. 14, the housing has an internal passage 44 that extends between the first and second ends 42, 43, such that, in use, airflow is permitted between the breathing device and the filter device 10 through the internal passage 44.

The second end 43 has an engagement portion 45 that allows selective engagement with the breathing device. The engagement portion is in the form of a track 45 on an internal surface of the side wall 41 which cooperates with a corresponding projection 46 on the breathing device. The track 45 has a first section 45a that extends along a direction substantially parallel to the longitudinal extent of the housing and a second section 45b that extends from the first section 45a in a direction transverse to the longitudinal extent of the housing. As such, the track 45 is angled, i.e., has an L-shaped appearance when viewed in section. Whilst in this embodiment, the track 45 has been described as being L-shaped, it is envisaged that any other curved or bent shape may apply.

As an alternative, the engagement portion may be a projection that cooperates with a track on a breathing device. The track may have an L-shaped appearance when viewed in section.

In another alternative, the engagement portion may be a helical groove or projection that forms a threaded connection with a corresponding helical groove or projection on a breathing device.

In the presently described embodiment, the track 45 is an opening that extends through the side wall 41. However, it can be appreciated that in other forms, the track 45 may be a recess into an internal surface the side wall 41.

In use, the first end 42 of the adaptor 40 is connected to the inlet spigot 20 of the filter device 10. The first end 42 is connected to the inlet spigot 20 via a friction fit. For example, the first end 42 can be dimensioned such that it can be inserted inside the opening 14 of the inlet spigot 20 in a force fitting (i.e., interference fit) relationship. In another example, the first end 42 can be dimensioned such that the spigot 20 can be inserted into the internal passage 44 of the adaptor in a force fitting (i.e., interference fit) relationship.

Once the adaptor 40 and filter device 10 are connected, the adaptor 40 can be releasably connected to the breathing device.

FIGS. 16A-16C show the steps involved in releasably connecting the adaptor 40 to the breathing device. These steps involve:
1. moving the adaptor 40 in a direction parallel to the longitudinal extent of the housing (see arrow in FIG. 16A) such that the projection 46 of the breathing device enters the first section 45a of the track 45, as shown in FIG. 16A;
2. moving the adaptor 40 in a direction parallel to the longitudinal extent of the housing (see arrow in FIG. 16A) such that the projection 46 travels towards the end of the first section 45a of the track; and
3. once the projection 46 reaches the end of the first section 45a of the track 45, as shown in FIG. 16B, rotating the housing in an anti-clockwise direction (when viewed from second end 43) such that the projection enters and travels along the second end 45b of the track 45, as shown in FIG. 16C.

As can be appreciated from the above, the selective engagement of the adaptor and breathing device involves an insertion and twisting motion, akin to a bayonet connection.

The adaptor 40 can be disconnected from the breathing device by following steps 1-3 in a reverse order, i.e. 3-1.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

What is claimed is:

1. A filter device for a breathing device, the filter device comprising: a housing with a wall made from a paper material and defining a volume, the housing comprising a first shell half defining a part of the wall and a second shell half defining another part of the wall that are releasably coupled together, two openings in the wall that communicate with the volume, and a filter mounting portion configured for mounting a filter media in the volume between the openings, wherein the filter mounting portion comprises a first projection on the part of the wall on the first shell half and a second projection on another part of the wall on the second shell half, each projection extending inwardly into the volume from respective parts of the wall, the projections being configured to frictionally engage the filter media therebetween when the first and second shell halves are coupled together to thereby support the filter media in the housing.

2. The filter device of claim 1, wherein the wall is indented inwardly into the volume to form the projections.

3. The filter device of claim 1, comprise engagement elements for releasably coupling together the shell halves, and wherein the engagement elements comprise a male element and a female element, the male element being configured to be inserted inside the female element in a friction fit arrangement.

4. The filter device of claim 3, wherein the engagement elements comprise a channel and a lip.

5. The filter device of claim 1, wherein the first shell half comprises an inlet configured for attachment to the breathing device.

6. The filter device of claim 1, wherein the second shell half comprises an outlet configured to conform to a mouth of a user.

7. A filter assembly comprising the filter device as defined in claim 1 and a filter media mounted to the filter mounting portion within the housing.

8. A method of manufacturing a filter device for a breathing device, the filter device comprising: a housing with a wall made from a paper material and defining a volume, the housing comprising a first shell half defining a part of the wall and a second shell half defining another part of the wall that are releasably coupled together, two openings in the wall that communicate with the volume and a filter mounting portion configured for mounting a filter media in the volume between the openings, wherein the filter mounting portion comprises a first projection on the part of the wall on the first shell half and a second projection on another part of the wall on the second shell half, each projection extending inwardly into the volume from respective parts of the wall, the projections being configured to frictionally engage the filter media therebetween when the first and second shell halves are coupled together to thereby support the filter media in the housing, the method of manufacturing comprising:
molding a paper material into a shape of the housing in a mold;
drying the housing; and
removing the housing from the mold.

9. An adaptor for use with a filter device, the adaptor comprising an adaptor housing having a first end that is configured for attachment with the filter device, a second end configured for attachment with a breathing device, and an internal passage that extends between the first and second ends, such that, in use, airflow is permitted between the breathing device and the filter device through the internal passage.

10. The adaptor of claim 9, wherein the adaptor housing is made from a paper material.

11. The adaptor of claim 9, wherein the second end has an engagement portion that allows releasable connection with the breathing device.

12. The adaptor of claim 11, wherein releasable connection between the second end and the breathing device involves an insertion and twisting motion.

13. The adaptor of claim 12, wherein the adaptor housing comprises a cylindrical wall defining an internal surface and external surface.

14. The adaptor of claim 13, wherein the engagement portion comprises a track or projection on the internal surface or the external surface that cooperates with a corresponding track or projection on the breathing device.

15. The adaptor of claim 14, wherein the track is curved or bent such that releasable connection between the second end and the breathing device involves the insertion and twisting motion.

16. The filter device of claim 1, wherein the filter device is coupled to an adaptor comprising an adaptor housing having a first end that is configured for attachment with the filter device, a second end configured for attachment with a breathing device, and an internal passage that extends between the first and second ends, such that, in use, airflow is permitted between the breathing device and the filter device through the internal passage.
